(19) United States
(12) Patent Application Publication
(10) Pub. No.: US 2026/0021080 A1
(43) Pub. Date: Jan. 22, 2026

(54) PHARMACEUTICAL COMPOUNDS, PHARMACEUTICAL COMPOSITIONS, AND METHODS OF TREATING DEPRESSION AND OTHER DISORDERS

(71) Applicant: Miralogx LLC, Tampa, FL (US)

(72) Inventor: Jonnie R. Williams, SR., Sarasota, FL (US)

(21) Appl. No.: 19/341,358

(22) Filed: Sep. 26, 2025

Related U.S. Application Data
(63) Continuation of application No. PCT/US2024/020832, filed on Mar. 21, 2024.
(60) Provisional application No. 63/454,867, filed on Mar. 27, 2023.
(60) Provisional application No. 63/524,378, filed on Jun. 30, 2023.

(51) Int. Cl. A61K 31/437; A61K 31/407; A61K 31/5355; C07D 457/14; C07D 487/06

(52) U.S. Cl. CPC A61K 31/437; A61K 31/407; A61K 31/5355; C07D 457/14; C07D 487/06

(57) ABSTRACT

In some aspects, pharmaceutical compounds are disclosed which may be useful for treating anxiety and/or other disorders. In another aspect, a pharmaceutical composition includes a therapeutically effective amount of the compound(s) and a pharmaceutically acceptable vehicle therefor. In yet another aspect, a method of treating anxiety, depression, obsessive-compulsive disorder, tobacco addiction, alcohol addiction, cocaine addiction, headache, cluster headache, or cancer-related or other end-of-life psychological distress involves administering the pharmaceutical composition to an individual in need thereof.

### CROSS-REFERENCE TO RELATED APPLICATIONS

This application is a continuation of International Application No. PCT/US2024/020832, filed Mar. 21, 2024, which claims priority under 35 U.S.C. § 119 (e) to U.S. App. No. 63/454,867, filed Mar. 27, 2023 and U.S. App. No. 63/524,378, filed Jun. 30, 2023, the disclosures of each of which are hereby incorporated by reference in their entireties.

### BACKGROUND

Lysergic acid diethylamide (LSD) is a hallucinogenic drug. Effects typically include altered thoughts, feelings, and awareness of one's surroundings. Many users have visual or auditory hallucinations. Dilated pupils, increased blood pressure, and increased body temperature are typical. Effects typically begin within half an hour and can last for up to 12 hours. It is used mainly as a recreational drug or for spiritual reasons, but also has been investigated for a number of therapeutic uses.

The mode of action of LSD is not fully understood. It is thought to interact with the serotonin system by binding to and activating 5-hydroxytryptamine subtype 2 receptor (5-HT2), which interferes with inhibitory systems resulting in perceptual disturbances. It is amongst the most potent drugs known, being active at doses from about 20 micrograms. Adverse psychiatric reactions are possible, such as anxiety, paranoia, and delusions. Distressing flashbacks might occur in spite of no further use, a condition called hallucinogen persisting perception disorder. In pure form, LSD is clear or white in color, has no smell, and is crystalline.

2-bromo-LSD is a serotonin 5-HT2A receptor antagonist. The compound blocks the effects of LSD, but has no psychological effects on its own. A 1959 study reported that some tolerance does develop to 2-bromo-LSD, and prolonged administration does not cause a significant resistance to the effects of LSD. Some studies have reported 2-bromo-LSD as being effective for treating or preventing cluster headaches.

There remains a need for improved therapies for treating disorders such as depression, anxiety, headache, and related disorders. It would be particularly desirable to develop small-molecule compounds that can be prepared synthetically and which exhibit improved efficacy while avoiding one or more drawbacks associated with presently available therapies.

### SUMMARY

In one aspect, a compound has a structure of Formula (I):

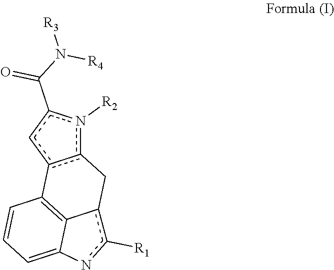

wherein R₁, R₂, R₃, and R₄ are each independently selected from the group consisting of an electron pair, H, halogen, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NRARB, —S-alkyl, —SO-alkyl, —SO₂-alkyl, alkenyl, alkynyl, aryl, heteroaryl, cycloalkyl, and heterocycle; wherein RA and RB are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NRCRD, —S-alkyl, —SO-alkyl and —SO₂-alkyl; wherein RC and RD are each independently selected from hydrogen and C₁₋₄ alkyl; each ______ represents a single or double bond, with the proviso that within a 5-membered ring, one ______ is a double bond and the other four ______ are single bonds; or a pharmaceutically acceptable salt or ester thereof. In some embodiments, R₃ and R₄, together with the nitrogen atom to which they are attached, form an optionally-substituted heterocyclic ring, e.g., heteroaryl or heterocycle.

In another aspect, a compound according to Formula (I) has a structure of Formula (Ia):

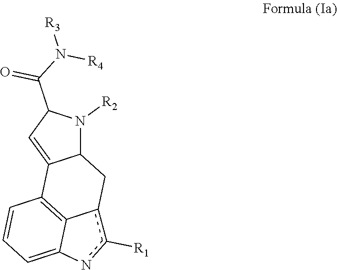

or a pharmaceutically acceptable salt or ester thereof.

In another aspect, a compound according to Formula (I) has a structure of Formula (Ib):

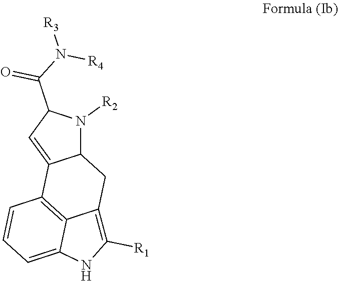

or a pharmaceutically acceptable salt or ester thereof.

In another aspect, the present disclosure relates to a compound according to Formula (I), (Ia), or (Ib), wherein R₃ and R₄, together with the nitrogen atom to which they are attached form an optionally-substituted heterocyclic ring; or a pharmaceutically acceptable salt or ester thereof.

In another aspect, the present disclosure relates to a compound according to Formula (I), (Ia), or (Ib), wherein R₁ is a halogen. In another aspect, the present disclosure relates to a compound according to Formula (I), (Ia), or (Ib), wherein the halogen is selected from the group consisting of F, Cl, Br, and I, preferably wherein the halogen is Br.

In another aspect, the present disclosure relates to a compound according to Formula (I), (Ia), or (Ib), wherein the compound is

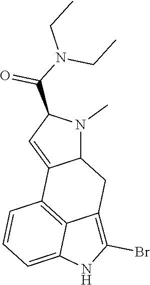 [SMILES: CCN(CC)C(=O)[C@@H]1C=C2c3cccc4[nH]c(Br)c(c34)CC2N1C]

or a pharmaceutically acceptable salt or ester thereof.

According to another aspect, a compound has a structure of Formula (II):

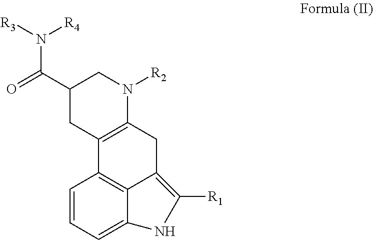

or a pharmaceutically acceptable salt, ester, or ether thereof, where R₁, R₂, R₃, and R₄ are as previously defined. In some embodiments, the present disclosure relates to a compound having the structure of Formula (II), wherein R₃ and R₄, together with the nitrogen atom to which they are attached form an optionally-substituted heterocyclic ring; or a pharmaceutically acceptable salt or ester thereof. In some embodiments, the present disclosure relates to a compound having the structure of Formula (II), wherein R₁ is a halogen. In some embodiments, the present disclosure relates to a compound having the structure of Formula (II), wherein the halogen is selected from the group consisting of F, Cl, Br, and I, preferably wherein the halogen is Br.

According to another aspect, a compound has a structure of Formula (III):

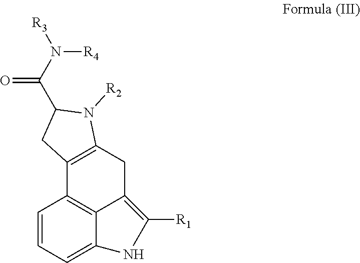

or a pharmaceutically acceptable salt, ester, or ether thereof, where R₁, R₂, R₃, and R₄ are as previously defined. In some embodiments, the present disclosure relates to a compound having the structure of Formula (III), wherein R₃ and R₄, together with the nitrogen atom to which they are attached form an optionally-substituted heterocyclic ring; or a pharmaceutically acceptable salt or ester thereof. In some embodiments, the present disclosure relates to a compound having the structure of Formula (III), wherein R₁ is a halogen. In some embodiments, the present disclosure relates to a compound having the structure of Formula (III), wherein the halogen is selected from the group consisting of F, Cl, Br, and I, preferably wherein the halogen is Br.

In another aspect, a pharmaceutical compound has a structure selected from the group consisting of:

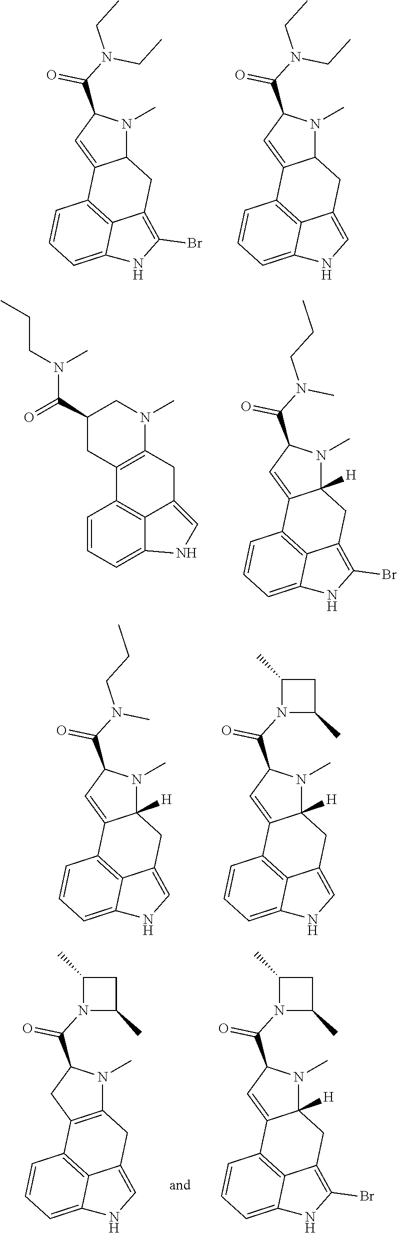

or a pharmaceutically acceptable salt, ester, or ether thereof.

In another aspect, a pharmaceutical composition comprises a therapeutically effective amount of a compound having at least one of the structures depicted above, and a pharmaceutically acceptable vehicle therefor.

In another aspect, a method of treating a disorder selected from the group consisting of anxiety, depression, obsessive-compulsive disorder, tobacco addiction, alcohol addiction, cocaine addiction, cluster headache, and psychological distress comprises administering to an individual in need thereof a therapeutically effective amount of a compound disclosed herein or a pharmaceutical composition disclosed herein.

### DETAILED DESCRIPTION

In one aspect, a compound has a structure of Formula (I):

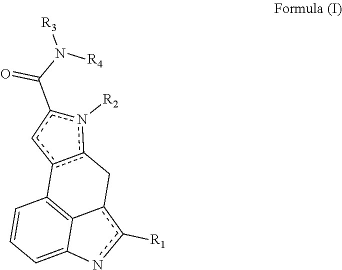

wherein R₁, R₂, R₃, and R₄ are each independently selected from the group consisting of an electron pair, H, halogen, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NRARB, —S-alkyl, —SO-alkyl, —SO₂-alkyl, alkenyl, alkynyl, aryl, heteroaryl, cycloalkyl, and heterocycle; wherein RA and RB are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NRCRD, —S-alkyl, —SO-alkyl and —SO₂-alkyl; wherein RC and RD are each independently selected from hydrogen and C₁₋₄ alkyl; each represents a single or double bond, with the proviso that within a 5-membered ring, one is a double bond and the other four are single bonds; or a pharmaceutically acceptable salt or ester thereof.

In some embodiments, R₃ and R₄, together with the nitrogen atom to which they are attached, form an optionally-substituted heterocyclic ring, e.g., heteroaryl or heterocycle.

In another aspect, a compound according to Formula (I) has a structure of Formula (Ia):

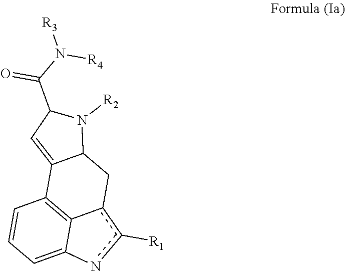

or a pharmaceutically acceptable salt or ester thereof.

In another aspect, a compound according to Formula (I) has a structure of Formula (Ib):

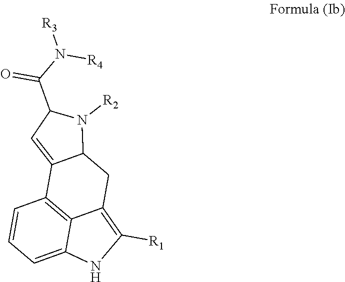

or a pharmaceutically acceptable salt or ester thereof.

In another aspect, R₃ and R₄, together with the nitrogen atom to which they are attached form an optionally-substituted heterocyclic ring; or a pharmaceutically acceptable salt or ester thereof. In another aspect, R₁ is a halogen. In another aspect, the halogen is selected from the group consisting of F, Cl, Br, and I, preferably wherein the halogen is Br.

In another aspect, the present disclosure relates to a compound according to Formula (I), (Ia), or (Ib), wherein the compound is

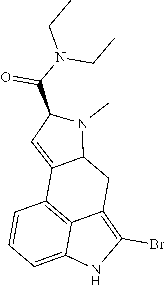 [SMILES: CCN(CC)C(=O)[C@@H]1C=C2c3cccc4[nH]c(Br)c(c34)CC2N1C]

or a pharmaceutically acceptable salt or ester thereof.

In one aspect, a compound has a structure of Formula (II):

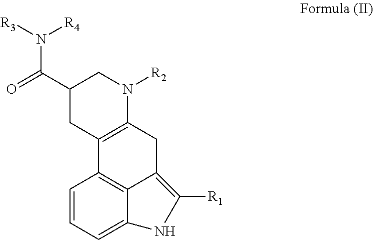

or a pharmaceutically acceptable salt, ester, or ether thereof, where R₁, R₂, R₃, and R₄ are as previously defined.

In another aspect, a pharmaceutical compound has a structure of Formula (II):

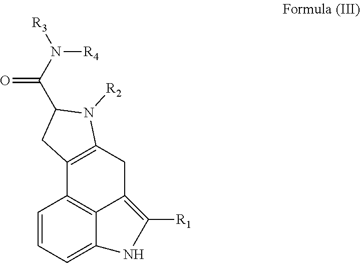

or a pharmaceutically acceptable salt, ester, or ether thereof, where R₁, R₂, R₃, and R₄ are as previously defined.

In another aspect, a compound has a structure selected from the group consisting of:

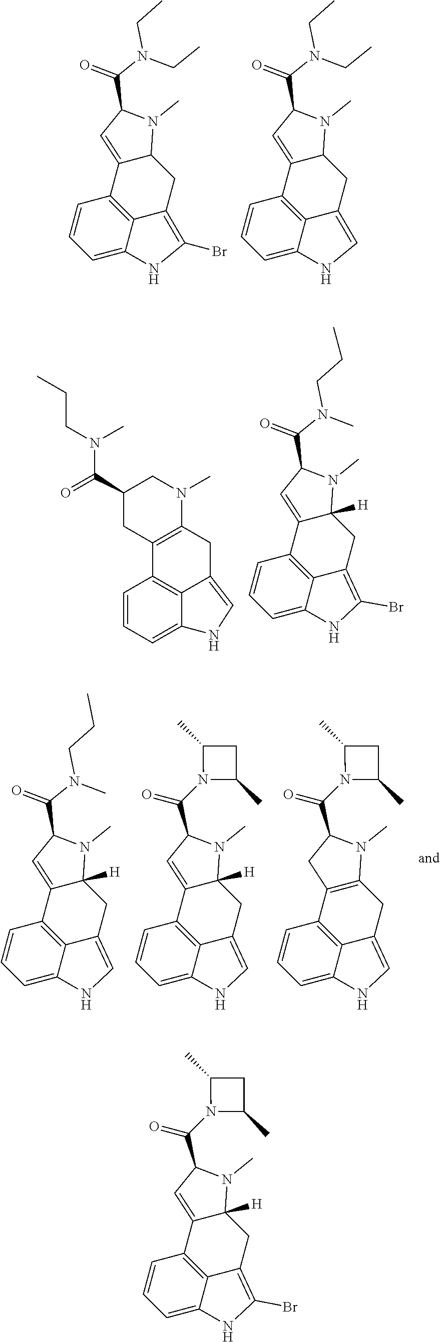

or a pharmaceutically acceptable salt, ester, or ether thereof.

In yet another aspect, a pharmaceutical compound has the structure:

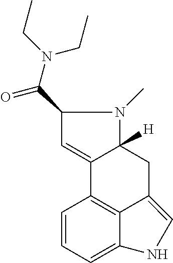 [SMILES: CCN(CC)C(=O)[C@@H]1C=C2c3cccc4[nH]cc(c34)C[C@H]2N1C]

or a pharmaceutically acceptable salt, ester, or ether thereof.

In still another aspect, a pharmaceutical compound has a structure selected from the group consisting of:

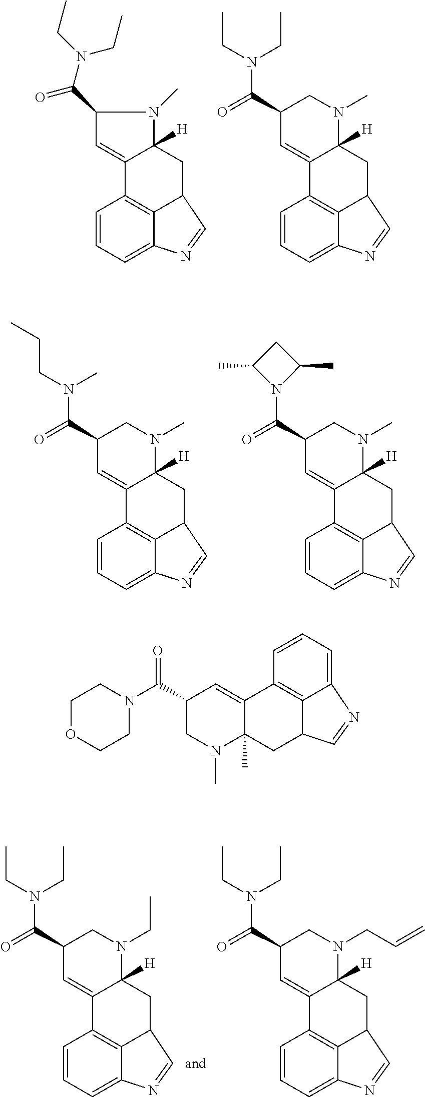

or a pharmaceutically acceptable salt, ester, or ether thereof.

A pharmaceutical composition may include a pharmaceutically acceptable carrier that facilitates processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle,” “stabilizer,” “diluent,” “additive,” “auxiliary” or “excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching herein.

As used herein the term “alkyl,” whether alone or as part of a substituent group, refers to a saturated C₁-Cₙ carbon chain, wherein the carbon chain may be straight or branched; wherein n can be 2, 3, 4, 5, 6, 7, 8, 9 or 10. Suitable examples include, but are not limited to methyl, ethyl, propyl, isopropyl, butyl, isobutyl, sec-butyl, t-butyl, n-pentyl and n-hexyl.

As used herein the term “alkenyl,” whether alone or as part of a substituent group, refers to a C₂-Cₙ carbon chain, wherein the carbon chain may be straight or branched, wherein the carbon chain contains at least one carbon-carbon double bond, and wherein n can be 3, 4, 5, 6, 7, 8, 9 or 10.

As used herein the term “alkynyl,” whether alone or as part of a substituent group, refers to a C₂-Cₙ, wherein the carbon chain may be straight or branched, wherein the carbon chain contains at least one carbon-carbon triple bond, and wherein n can be 3, 4, 5, 6, 7, 8, 9 or 10.

As used herein the term “aryl,” whether alone or as part of a substituent group, refers to an unsubstituted carboxylic aromatic ring comprising between 6 to 14 carbon atoms. Suitable examples include, but are not limited to, phenyl and naphthyl.

As used herein the term “protected hydroxyl” refers to a hydroxyl group substituted with a suitably selected oxygen protecting group. More particularly, a “protected hydroxyl” refers to a substituent group of the formula —OPG₁ wherein PG₁ is a suitably selected oxygen protecting group. During any of the processes for preparation of the compounds of the present disclosure it may be necessary and/or desirable to protect sensitive or reactive groups on any of the molecules concerned. This may be achieved by means of conventional protecting groups, such as those described in Protective Groups in Organic Chemistry, ed. J. F. W. McOmic, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein the term “oxygen protecting group” refers to a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM and THP. Other suitable oxygen protecting groups may be found in texts such as T. W. Greene & P. G. M. Wuts, Protective Groups in Organic Synthesis, John Wiley & Sons, 1991.

As used herein the term “nitrogen protecting group” refers to a group which may be attached to a nitrogen atom to protect said nitrogen atom from participating in a reaction and which may be readily removed following the reaction. Suitable nitrogen protecting groups include, but are not limited to, carbamates groups of the formula —C(O)—OR wherein R can be methyl, ethyl, t-butyl, benzyl, phenylethyl, CH₂═CH—CH₂—, and the like; amide groups of the formula —C(O)—R′ wherein R′ can be methyl, phenyl, trifluoromethyl, and the like; N-sulfonyl derivative groups of the formula —SO₂—R″ wherein R″ can be tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein the term “acyl” refers to a group of the formula —CO—Cₙ wherein Cₙ represent a straight or branched alkyl chain wherein n can be 1, 2, 3, 4, 5, 6, 7, 8, 9 or 10.

As used herein the term “heteroaryl” refers to any five or six membered monocyclic aromatic ring structure containing at least one heteroatom selected from the group consisting of O, N and S, and optionally containing one to three additional heteroatoms independently selected from the group consisting of O, N and S; or a nine or ten membered bicyclic aromatic ring structure containing at least one heteroatom selected from the group consisting of O, N and S, and optionally containing one to four additional heteroatoms independently selected from the group consisting of O, N and S. The heteroaryl group may be attached at any heteroatom or carbon atom of the ring such that the result is a stable structure. Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl and pteridinyl.

As used herein the term “cycloalkyl” refers to any monocyclic ring containing from four to six carbon atoms, or a bicyclic ring containing from eight to ten carbon atoms. The cycloalkyl group may be attached at any carbon atom of the ring such that the result is a stable structure. Examples of suitable cycloalkyl groups include, but are not limited to, cyclopropyl, cyclobutyl, cyclopentyl and cyclohexyl.

As used herein the term “heterocycle” refers to any four to six membered monocyclic ring structure containing at least one heteroatom selected from the group consisting of O, N and S, and optionally containing one to three additional heteroatoms independently selected from the group consisting of O, N and S; or an eight to ten membered bicyclic ring structure containing at least one heteroatom selected from the group consisting of O, N and S, and optionally containing one to four additional heteroatoms independently selected from the group consisting of O, N and S. The heterocycle group may be attached at any heteroatom or carbon atom of the ring such that the result is a stable structure. Examples of suitable heterocycle groups include, but are not limited to, azetidine, azete, oxetane, oxete, thietane, thiete, diazetidine, diazete, dioxetane, dioxete, dithietane, dithiete, pyrrolidine, pyrrole, tetrahydrofuran, furan, thiolane, thiophene, piperidine, oxane, thiane, pyridine, pyran and thiopyran.

The groups described herein can be unsubstituted or substituted, as herein defined. In addition, the substituted groups can be substituted with one or more groups such as a C₁-C₆ alkyl, C₁₋₄ alkyl, —O—C₁₋₄ alkyl, hydroxyl, amino, (C₁₋₄ alkyl) amino, di (C₁₋₄ alkyl) amino, —S—(C₁₋₄ alkyl), —SO—(C₁₋₄ alkyl), —SO₂—(C₁₋₄ alkyl), halogen, aryl, heteroaryl, and the like.

The term “halogen,” as used herein, refers to fluorine, chlorine, bromine, iodine, or astatine.

With reference to substituents, the term “independently” means that when more than one of such substituents is possible, such substituents may be the same or different from each other.

The compounds of the present disclosure may contain at least one hydroxyl group. These at least one hydroxyl group may form an ester with inorganic or organic acid. In particular, pharmaceutically acceptable acids. The ester(s) may form chiral carbons. The present disclosure is directed toward all stereo-chemical forms of the compounds of the present disclosure, including those formed by the formation of one or more ester groups.

Compounds intended for administration to humans or other mammals generally should have very high purity. Purity refers to the ratio of a compound's mass to the total sample mass following any purification steps. Usually, the level of purity is at least about 95%, more usually at least about 96%, about 97%, about 98%, or higher. For example, the level of purity may be about 98.5%, 99.0%, 99.1%, 99.2%, 99.3%, 99.4%, 99.5%, 99.6%, 99.7%, 99.8%, 99.9%, or higher.

The compounds described herein that exist in more than one optical isomer form (enantiomer) may be provided either as racemic mixture or by isolating one of the enantiomers, the latter case in which purity as described above may refer to enantiomeric purity.

The compounds may be prepared synthetically using techniques described, e.g., in Liu et al., “Total Synthesis of (+)-Lysergic Acid,” J. Org. Chem. 2013, 78, 21, 10885-10893 with appropriate modifications of reagents to obtain the structures described herein as will be apparent to persons skilled in the art.

In some aspects, the compounds may be converted into a pharmaceutically acceptable salts using techniques well known to persons skilled in the art. For example, salts such as sodium and potassium salts may be prepared by treating the compound with a suitable sodium or potassium base, such as sodium hydroxide or potassium hydroxide, respectively. Esters and ethers of the compounds may be prepared as described, e.g., in Advanced Organic Chemistry, 1992, 4th Edition, J. March, John Wiley & Sons, or J. Med. Chemistry, 1992, 35, 145-151.

The compounds as described herein may be useful for treating anxiety, depression, obsessive-compulsive disorder, tobacco addiction (including smoking addiction and smokeless tobacco addiction), alcohol addiction, cocaine addiction, headache (including cluster headache), and cancer-related or other end-of-life psychological distress.

Compositions as described herein may be administered orally, nasally, topically, subcutaneously, intramuscularly, intravenously, or by other suitable modes of administration.

A pharmaceutical composition may optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

Examples of auxiliaries and/or excipients that may be mentioned are cremophor, poloxamer, benzalkonium chloride, sodium lauryl sulfate, dextrose, glycerin, magnesium stearate, polyethylene glycol, starch, dextrin, lactose, cellulose, carboxymethylcellulose sodium, talc, agar-agar, mineral oil, animal oil, vegetable oil, organic and mineral waxes, paraffin, gels, propylene glycol, benzyl alcohol, dimethylacetamide, ethanol, polyglycols, tween 80, solutol HS 15, and water. It is also possible to administer the active substances as such, without vehicles or diluents, in a suitable form, for example, in capsules.

A pharmaceutical composition may comprise a therapeutic compound in an amount sufficient to allow customary administration to an individual. A unit dose form may have, e.g., at least 0.001 mg, at least 0.005 mg, at least 0.01 mg, at least 0.1 mg, at least 0.5 mg, at least 0.75 mg, at least 1 mg, at least 1.5 mg, at least 2 mg, at least 2.5 mg, at least 5 mg, at least 7.5 mg, at least 10 mg, at least 15 mg, at least 20 mg, at least 25 mg, at least 30 mg, at least 40 mg, or at least 50 mg of a therapeutic compound. In yet other aspects of this embodiment, a pharmaceutical composition disclosed herein may include, e.g., about 0.001 mg to about 500 mg, about 0.01 mg to about 250 mg, or about 0.05 mg to about 100 mg of a therapeutic compound.

Pharmaceutical compositions as described herein may include a pharmaceutically acceptable solvent. A solvent is a liquid, solid, or gas that dissolves another solid, liquid, or gaseous (the solute), resulting in a solution. Solvents useful in the pharmaceutical compositions include, without limitation, a pharmaceutically acceptable polar aprotic solvent, a pharmaceutically acceptable polar protic solvent and a pharmaceutically acceptable non-polar solvent. A pharmaceutically acceptable polar aprotic solvent includes, without limitation, dichloromethane (DCM), tetrahydrofuran (THF), ethyl acetate, acetone, dimethylformamide (DMF), acetonitrile (MeCN), dimethyl sulfoxide (DMSO). A pharmaceutically acceptable polar protic solvent includes, without limitation, acetic acid, formic acid, ethanol, n-butanol, 1-butanol, 2-butanol, isobutanol, sec-butanol, tert-butanol, n-propanol, isopropanol, 1,2 propan-diol, methanol, glycerol, and water. A pharmaceutically acceptable non-polar solvent includes, without limitation, pentane, cyclopentane, hexane, cyclohexane, benzene, toluene, 1,4-dioxane, chloroform, n-methyl-pyrrolidone (NMP), and diethyl ether.

The method of administration as well as the dosage range which are suitable in a specific case depend on the species to be treated and on the state of the respective condition or disease, and may be optimized using techniques known in the art. In some aspects, microdosing techniques may be used as described, e.g., in L.P. Cameron et al., “Chronic, Intermittent Microdoses of the Psychedelic N,N-Dimethyltryptamine (DMT) Produce Positive Effects on Mood and Anxiety in Rodents,” ACS Chem. Neurosci. 2019, 10, 3261-70.

By way of non-limiting example, the daily dose of an active compound may be about 0.0001 mg to about 10 mg per kg, or from about 0.0005 mg to about 5 mg per kg. Dosing can be single dosage or cumulative (serial dosing), and can be readily determined by one skilled in the art. For instance, treatment of anxiety may comprise a one-time administration of an effective dose of a pharmaceutical composition as disclosed herein. Alternatively, treatment may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

Pharmaceutical compositions may contain any conventional non-toxic pharmaceutically acceptable carriers, adjuvants or vehicles. In some cases, the pH of the formulation may be adjusted with acceptable pharmaceutical or food grade acids, bases or buffers to enhance the stability of the formulated composition or its delivery form.

Liquid dosage forms for oral administration include acceptable pharmaceutical or food grade emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylsulfoxide (DMSO) dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, lozenges, pills, powders, and granules. In such solid dosage forms, the active compound is mixed with at least one inert, acceptable pharmaceutical or food grade excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidone, sucrose and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as cetyl alcohol and glycerol monostearate, h) absorbents such as kaolin and bentonite clay, i) lubricants such as talc, calcium stearate, magnesium stearate, solid polyethylene glycols, sodium lauryl sulfate, and mixtures thereof, and j) sweetening, flavoring, perfuming agents, and mixtures thereof. In the case of capsules, lozenges, tablets and pills, the dosage form may also comprise buffering agents.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract or, optionally, in a delayed or extended manner. Examples of embedding compositions which can be used include polymeric substances and waxes. Tablet formulations for extended release are also described in U.S. Pat. No. 5,942,244.

Compositions may contain a compound as disclosed herein, alone or with other therapeutic compound(s). A therapeutic compound is a compound that provides pharmacological activity or other direct effect in the diagnosis, cure, mitigation, treatment, or prevention of disease, or to affect the structure or any function of the body of man or animals. A therapeutic compound disclosed herein may be used in the form of a pharmaceutically acceptable salt, solvate, or solvate of a salt, e.g., a hydrochloride. Additionally, therapeutic compound disclosed herein may be provided as racemates, or as individual enantiomers, including the R- or S-enantiomer. Thus, the therapeutic compound disclosed herein may comprise a R-enantiomer only, a S-enantiomer only, or a combination of both a R-enantiomer and a S-enantiomer of a therapeutic compound. In some aspects, the therapeutic compound may have anti-inflammatory activity, such as a non-steroidal anti-inflammatory drug (NSAID). NSAIDs are a large group of therapeutic compounds with analgesic, anti-inflammatory, and anti-pyretic properties. NSAIDs reduce inflammation by blocking cyclooxygenase. NSAIDs include, without limitation, aceclofenac, acemetacin, actarit, alcofenac, alminoprofen, amfenac, aloxipirin, aminophenazone, antraphenine, aspirin, azapropazone, benorilate, benoxaprofen, benzydamine, butibufen, celecoxib, chlorthenoxacin, choline salicylate, clometacin, dexketoprofen, diclofenac, diflunisal, emorfazone, epirizole; ctodolac, etoricoxib, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, lumiracoxib, mefenamic acid, meloxicam, metamizole, metiazinic acid, mofebutazone, mofezolac, nabumetone, naproxen, nifenazone, niflumic acid, oxametacin, phenacetin, pipebuzone, pranoprofen, propyphenazone, proquazone, protizinic acid, rofecoxib, salicylamide, salsalate, sulindac, suprofen, tiaramide, tinoridine, tolfenamic acid, valdecoxib, and zomepirac.

NSAIDs may be classified based on their chemical structure or mechanism of action. Non-limiting examples of NSAIDs include a salicylate derivative NSAID, a p-amino phenol derivative NSAID, a propionic acid derivative NSAID, an acetic acid derivative NSAID, an enolic acid derivative NSAID, a fenamic acid derivative NSAID, a non-selective cyclooxygenase (COX) inhibitor, a selective cyclooxygenase-1 (COX-1) inhibitor, and a selective cyclooxygenase-2 (COX-2) inhibitor. An NSAID may be a profen. Examples of a suitable salicylate derivative NSAID include, without limitation, acetylsalicylic acid (aspirin), diflunisal, and salsalate. Examples of a suitable p-amino phenol derivative NSAID include, without limitation, paracetamol and phenacetin. Examples of a suitable propionic acid derivative NSAID include, without limitation, alminoprofen, benoxaprofen, dexketoprofen, fenoprofen, flurbiprofen, ibuprofen, indoprofen, ketoprofen, loxoprofen, naproxen, oxaprozin, pranoprofen, and suprofen. Examples of a suitable acetic acid derivative NSAID include, without limitation, aceclofenac, acemetacin, actarit, alcofenac, amfenac, clometacin, diclofenac, ctodolac, felbinac, fenclofenac, indometacin, ketorolac, metiazinic acid, mofezolac, nabumetone, naproxen, oxametacin, sulindac, and zomepirac. Examples of a suitable enolic acid (oxicam) derivative NSAID include, without limitation, droxicam, isoxicam, lornoxicam, meloxicam, piroxicam, and tenoxicam. Examples of a suitable fenamic acid derivative NSAID include, without limitation, flufenamic acid, mefenamic acid, meclofenamic acid, and tolfenamic acid. Examples of a suitable selective COX-2 inhibitors include, without limitation, celecoxib, etoricoxib, firocoxib, lumiracoxib, meloxicam, parecoxib, rofecoxib, and valdecoxib.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. While advantages associated with certain embodiments have been described in the context of these embodiments, other embodiments may also exhibit such advantages, and not all embodiments need necessarily exhibit such advantages to fall within the scope of the disclosure.

While the invention has been described with respect to specific examples, those skilled in the art will appreciate that there are numerous variations and permutations of the above described systems and techniques that fall within the spirit and scope of the invention as set forth in the appended claims.

What is claimed is:

1. A compound having a structure of Formula (I): 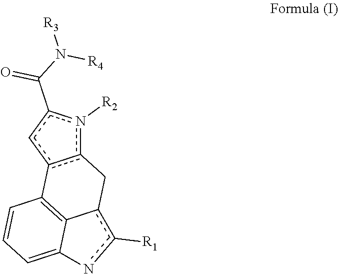
wherein R₁, R₂, R₃, and R₄ are each independently selected from the group consisting of an electron pair, H, halogen, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NRARB, —S-alkyl, —SO-alkyl, —SO₂-alkyl, alkenyl, alkynyl, aryl, heteroaryl, cycloalkyl, and heterocycle; wherein RA and R_{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NRCRD, —S-alkyl, —SO-alkyl and —SO₂-alkyl; wherein RC and RD are each independently selected from hydrogen and C₁₋₄ alkyl; each ______ represents a single or double bond, with the proviso that within a 5-membered ring, one ______ is a double bond and the other four ______ are single bonds; or
a pharmaceutically acceptable salt or ester thereof.

2. The compound of claim 1, wherein the compound has a structure of Formula (Ia): 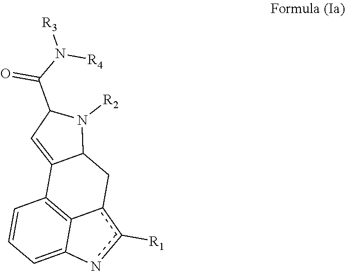
or a pharmaceutically acceptable salt or ester thereof.

3. The compound of claim 1, wherein the compound has a structure of Formula (Ib): 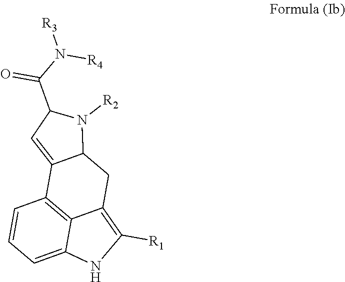
or a pharmaceutically acceptable salt or ester thereof.

4. The compound of claim 1, wherein R₃ and R₄, together with the nitrogen atom to which they are attached form an optionally-substituted heterocyclic ring; or a pharmaceutically acceptable salt or ester thereof.

5. The compound of claim 1, wherein R₁ is a halogen.

6. The compound of claim 5, wherein the halogen is selected from the group consisting of F, Cl, Br, and I, preferably wherein the halogen is Br.

7. The compound of claim 1, wherein the compound is 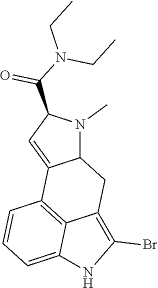 [SMILES: CCN(CC)C(=O)[C@@H]1C=C2c3cccc4[nH]c(Br)c(c34)CC2N1C]
or a pharmaceutically acceptable salt or ester thereof.

8. A compound having a structure of Formula (II): 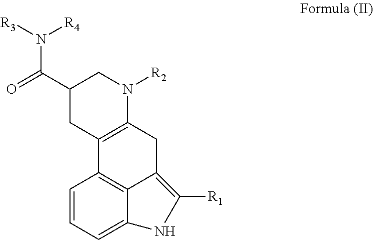
wherein R₁, R₂, R₃, and R₄ are each independently selected from the group consisting of an electron pair, H, halogen, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NRARB, —S-alkyl, —SO-alkyl, —SO₂-alkyl, alkenyl, alkynyl, aryl, heteroaryl, cycloalkyl, and heterocycle; wherein RA and RB are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O) O—C₁₋₄ alkyl, NRCRD, —S-alkyl, —SO-alkyl and —SO₂-alkyl; wherein RC and RD are each independently selected from hydrogen and C₁₋₄ alkyl; or a pharmaceutically acceptable salt, ester, or ether thereof.

9. The compound of claim 8, wherein R₃ and R₄, together with the nitrogen atom to which they are attached form an optionally-substituted heterocyclic ring; or a pharmaceutically acceptable salt or ester thereof.

10. The compound of claim 9, wherein R₁ is a halogen.

11. The compound of claim 10, wherein the halogen is selected from the group consisting of F, Cl, Br, and I.

12. A compound of claim 1 which has a structure of Formula (III): 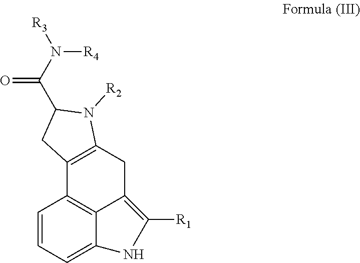
or a pharmaceutically acceptable salt, ester, or ether thereof.

13. The compound of claim 12, wherein R₃ and R₄, together with the nitrogen atom to which they are attached form an optionally-substituted heterocyclic ring; or a pharmaceutically acceptable salt or ester thereof.

14. The compound of claim 13, wherein R₁ is a halogen.

15. The compound of claim 14, wherein the halogen is selected from the group consisting of F, Cl, Br, and I.

16. A compound of claim 1 which has a structure selected from the group consisting of: 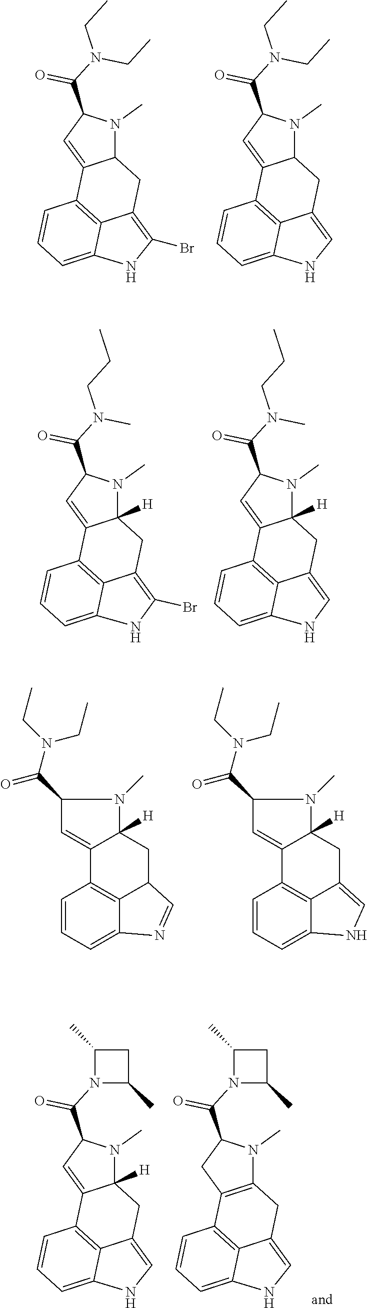 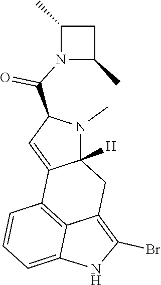 [SMILES: C[C@@H]1C[C@@H](C)N1C(=O)[C@@H]1C=C2c3cccc4[nH]c(Br)c(c34)C[C@H]2N1C]
or a pharmaceutically acceptable salt, ester, or ether thereof.

17. A compound having a structure selected from the group consisting of: 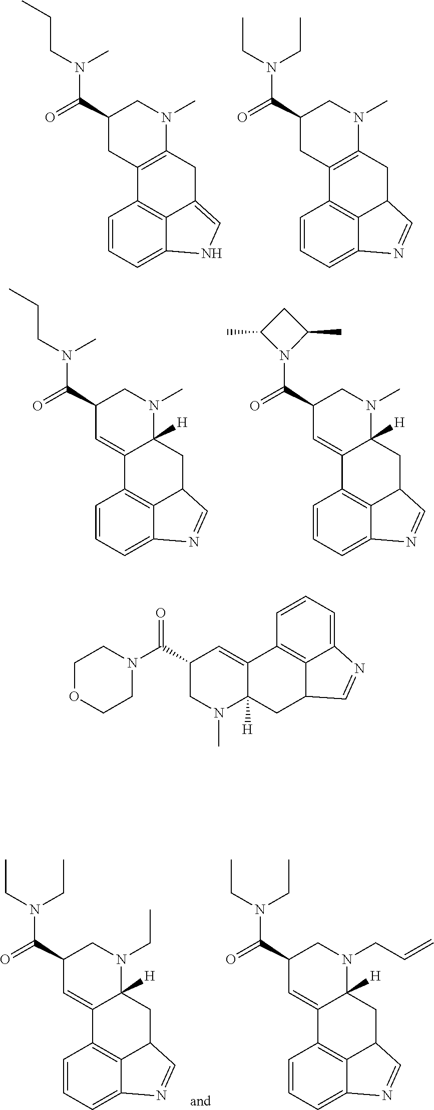
or a pharmaceutically acceptable salt, ester, or ether thereof.

18. A pharmaceutical composition comprising a therapeutically effective amount of a compound of claim 16 and a pharmaceutically acceptable vehicle therefor.

19. A method of treating anxiety comprising administering to an individual in need thereof the pharmaceutical composition according to claim 18.

20. A method of treating a disorder selected from the group consisting of depression, obsessive-compulsive disorder, tobacco addiction, alcohol addiction, cocaine addiction, headache, and psychological distress, comprising administering to an individual in need thereof the pharmaceutical composition of claim 18.
